(19) United States
(12) Patent Application Publication
(10) Pub. No.: US 2009/0312843 A1
(43) Pub. Date: Dec. 17, 2009

(54) Tissue Reconstruction Devices and Methods

(76) Inventors: Steven Palmer Ford, Newbury Park, NC (US); Palmer Ford, Carlsbad, CA (US)

Correspondence Address: FISH & ASSOCIATES, PC;ROBERT D. FISH, 2603 Main Street, Suite 1000, Irvine, CA, 92614-6232, US

(21) Appl. No.: 12/422,168

(22) Filed: Apr. 10, 2009

Related U.S. Application Data
(60) Provisional application No. 61/044,421, filed on Apr. 11, 2008.

(51) Int. Cl. A61F 2/02; B32B 37/12

(52) U.S. Cl. 623/23.72; 606/151; 156/293

(57) ABSTRACT

The present invention provides an implantable prosthesis comprising a viscera separating barrier that defines a shell having a pocket with an opening that receives an insert. It is contemplated that the insert comprises at least one of an absorbable mesh, a non-absorbable mesh, or an absorbable and non-absorbable mesh combination inserted into the pocket through the opening. In preferred embodiments the viscera separating barrier is acellular collagen, and the absorbable mesh is selected from at least one of a polyglactin, a polyglycolic acid, a polyglactin and a polylactic acid, and the non-absorbable mesh comprises a polypropylene mesh. It is contemplated that the viscera separating barrier acts to reduce or even eliminate attachment of viscera to the prosthesis to encourage rapid cell penetration and revascularization; the non-absorbable mesh will provide immediate, reliable in-growth; and the absorbable mesh will give stability to the prosthesis.

### PRIORITY STATEMENT

This application claims priority to U.S. Provisional Application No. 61/044,421 filed Apr. 11, 2008 which is incorporated by reference herein in its entirety.

### FIELD OF THE INVENTION

The field of the invention is implantable tissue supports.

### BACKGROUND

Implantable tissue supports are used to repair and reinforce anatomic defects or weaknesses. Common uses include hernia repair (inguinal and ventral), vaginal slings, pelvic floor reconstruction, plastic surgery, dural repair, rotator cuff repair, and staple line reinforcement.

In each of these situations it is desirable to use materials that provide for both long-term ingrowth from juxtaposed tissue and immediate strength. While both purposes can be achieved using only a single material, such as a polypropylene mesh (PPM), the use of PPM alone tends to cause undesired adhesions, severe scarring and potential for erosion into adjacent structures. Collagen based materials are also used for tissue reconstruction due to their resistance to infection and ability to encourage rapid cell penetration and revascularization. However, these materials have high failure rates because they tend to lose tensile strength and stretch as they incorporate into host tissue. Absorbable meshes are only indicated to provide temporary support and cannot be utilized to repair anatomic defects or weaknesses. One solution is to utilize a patch with multiple different layers.

The Avaulta Plus™ Biosynthetic Support System, for example, utilizes a two layer structure having an acellular collagen layer that minimizes attachment to viscera, and a nonabsorbable monofilament, polypropylene mesh that provides for both ingrowth and long-term strength. U.S. Pat. No. 7,101,381 to Ford et al (September 2006) describes an implantable structure having an underlay and an overlay coupled by two arms of a tether. The underlay has two ingrowth layers that are stitched or heat-bonded together. Ford's ingrowth layers are contemplated to comprise any of numerous materials, including non-absorbable meshes such as BARD MESH™ (polypropylene mesh from C.R. Bard, Inc.), SOFT TISSUE PATCH™ (microporous ePTFE available from W.L. Gore & Associates, Inc.), SURGIPRO™ (available from US Surgical, Inc.), TRELEX™ (available from Meadox Medical), PROLENE™ and MERSILENE™ (available from Ethicon, Inc.); absorbable materials such as VICRYL™ (polyglactin available from Ethicon, Inc.), DEXON™ (polyglycolic acid available from US Surgical, Inc.); and collagen materials such as COOK SURGISIS™ (collagen sheet available from Cook Biomedical, Inc.).

The '381 patent and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

One difficulty with conventional devices is that different types of materials have not been combined because they cannot practically be sterilized together. Thus, although the ingrowth layers in the '381 patent are described as being selected from any of multiple materials, and the ingrowth layers can comprise different materials from the tether, there is no indication that the two tissue-infiltratable layers can comprise different types of materials from one another.

It would be especially beneficial to provide a patch that can combine: (a) a non-absorbable polypropylene mesh for immediate, reliable in-growth; (b) a collagen mesh that reduces attachment to the viscera, encourages rapid cell penetration and revascularization; and (c) an absorbable mesh that gives stability to the patch and separates the PPM from the collagen mesh and can be used to host active materials such as growth factors and/or stem cells.

### SUMMARY OF THE INVENTION

The present invention provides apparatus, systems and methods in which an implantable prosthesis comprises a viscera separating barrier that defines a shell having a pocket with an opening that receives an insert. It is contemplated that the insert comprises at least one of an absorbable mesh, a non-absorbable mesh, or an absorbable and non-absorbable mesh combination inserted into the pocket through the opening.

In preferred embodiments the viscera separating barrier is acellular collagen, and independently the absorbable mesh is selected from at least one of a polyglactin, a polyglycolic acid (PGA), a polyglactin and a polylactic acid (PLA), and the non-absorbable mesh comprises a polypropylene mesh. The absorbable and non-absorbable meshes are preferably sewn or otherwise coupled together, and can include a stiffening ring. One or more of the viscera separating barrier, absorbable mesh, and non-absorbable mesh can include growth factors and/or stem cells.

While not wishing to be limited to any particular theory or method of action, it is contemplated that the viscera separating barrier will act to reduce or even eliminate attachment of viscera to the prosthesis to encourage rapid cell penetration and revascularization; the non-absorbable mesh will provide immediate, reliable in-growth, and the absorbable mesh will give stability to the prosthesis, and separate the PPM from the collagen mesh.

In situations where complete resorption of the prosthesis is desired (for example pediatric patients where PPM is contra-indicated), a viscera separating barrier can be coupled with a completely absorbable mesh. In this scenario, the absorbable will support the collagen network as it is incorporating into host tissue, reducing the chance of prosthesis failure or stretching.

Various objects, features, aspects and advantages of the inventive subject matter will become more apparent from the following detailed description of preferred embodiments, along with the accompanying drawings in which like numerals represent like components.

### BRIEF DESCRIPTION OF THE DRAWINGS

FIG. 1A is a presentation slide depicting an implantable prosthesis that defines a shell having a pocket with an opening that receives an insert.

FIG. 1B is a presentation slide that depicts an embodiment of the shell without the insert.

FIG. 1C is a presentation slide that depicts the shell having the insert introduced within the shell.

FIG. 2A is a presentation slide depicting a second alternative implantable prosthesis that defines a shell having a pocket, anterior holes, and a slit for inserting the insert.

FIG. 2B is a presentation slide depicting an elevated cross-sectional view of the prosthesis of FIG. 2A having anterior holes.

FIG. 3A is a presentation slide depicting a third alternative implantable prosthesis that defines a shell having a pocket, anterior and posterior holes, and a slit for inserting the insert.

FIG. 3B is a presentation slide depicting an elevated cross-sectional view of the prosthesis of FIG. 3A having anterior and posterior holes.

FIG. 4A is a presentation slide depicting a fourth alternative implantable prosthesis that defines a shell having a slit for inserting the insert.

FIG. 4B is a presentation slide depicting an elevated cross-sectional view of the prosthesis of FIG. 4A.

FIG. 5A is a presentation slide depicting a fifth alternative implantable prosthesis that defines a shell, having an injection port, and posterior fixation points.

FIG. 5B is a presentation slide depicting a cross-sectional view of the prosthesis of FIG. 5A.

FIG. 6A is a presentation slide describing manufacturing details of the viscera separating barrier.

FIG. 6B is a presentation slide describing the viscera separating barrier and the use of a femtosecond laser.

FIG. 6C is a presentation slide describing additional details of the use of a femtosecond laser and the viscera separating barrier.

FIG. 6D is a presentation slide depicting a viscera separating barrier cut by a femtosecond laser.

FIG. 7A is a presentation slide describing an insert (i.e. mesh inlay) that is inserted into the viscera separating barrier.

FIG. 7B is a presentation slide describing a mesh inlay edge stiffener.

FIG. 7C is a presentation slide describing additional details of the mesh inlay edge stiffener.

FIG. 8 is a presentation slide depicting a kit and corresponding instructions that can be used in a marketing program for the implantable prosthesis.

FIG. 9A is a presentation slide depicting steps in the treatment of a Laparoscopic Ventral Hernia (LVH) using devices and methods disclosed herein.

FIG. 9B is a presentation slide depicting additional steps in the treatment of a Laparoscopic Ventral Hernia (LVH) using devices and methods disclosed herein.

FIG. 9C is a presentation slide depicting steps in the treatment of an Open Ventral Hernia Repair (OVH) using devices and methods disclosed herein.

FIG. 9D is a presentation slide depicting additional steps in the treatment of an Open Ventral Hernia Repair (OVH) using devices and methods disclosed herein.

FIG. 10 is a presentation slide depicting steps in an inguinal hernia repair using devices and methods disclosed herein

FIG. 11A is a presentation slide depicting the contemplated progression of tissue growth into a prosthesis as disclosed herein—several days after surgery.

FIG. 11B is a presentation slide depicting the contemplated progression of tissue growth into a prosthesis as disclosed herein—10 days after surgery

FIG. 11C is a presentation slide depicting the contemplated progression of tissue growth into a prosthesis as disclosed herein—90 days after surgery.

FIG. 12 is a presentation slide listing 15 procedures for which specific prosthesis designs are contemplated.

### DETAILED DESCRIPTION

In the following description, reference is made to the accompanying drawings that form a part hereof, and in which is shown, by way of illustration, specific embodiments in which the invention may be practiced. These embodiments are described in sufficient detail to enable those skilled in the art to practice the invention, and it is to be understood that other embodiments may be utilized and that changes may be made without departing from the scope of the present invention. The following description is, therefore, not to be taken in a limited sense, and the scope of the present invention is defined by the appended claims.

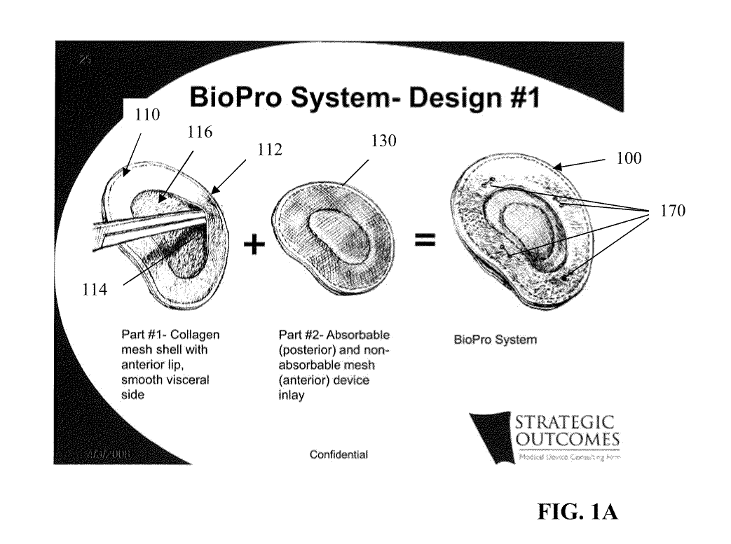

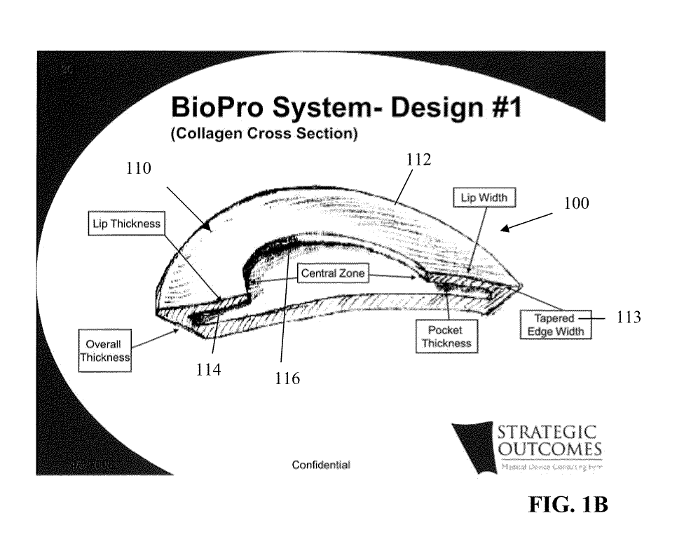

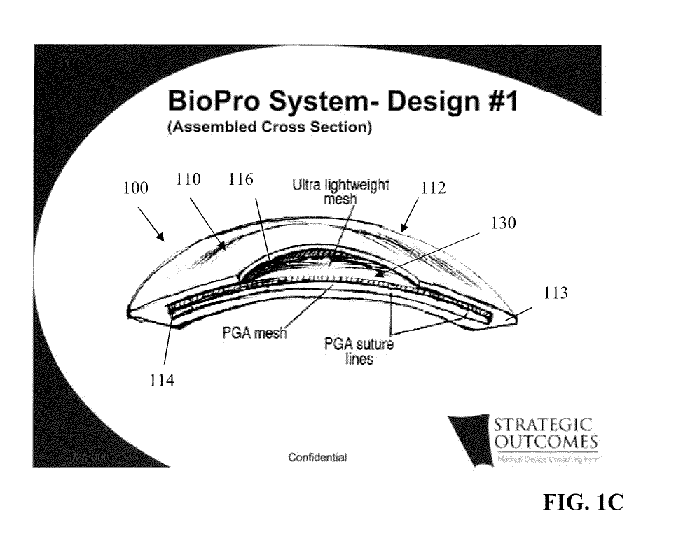

FIGS. 1A, 1B, and 1C generally depict an implantable prosthesis 100 having a viscera separating barrier 110 that defines a shell 112 having a pocket 114 with an opening 116 that receives an insert 130 that comprises at least one of an absorbable mesh, a non-absorbable mesh, or an absorbable and non-absorbable mesh combination inserted into the pocket through the opening.

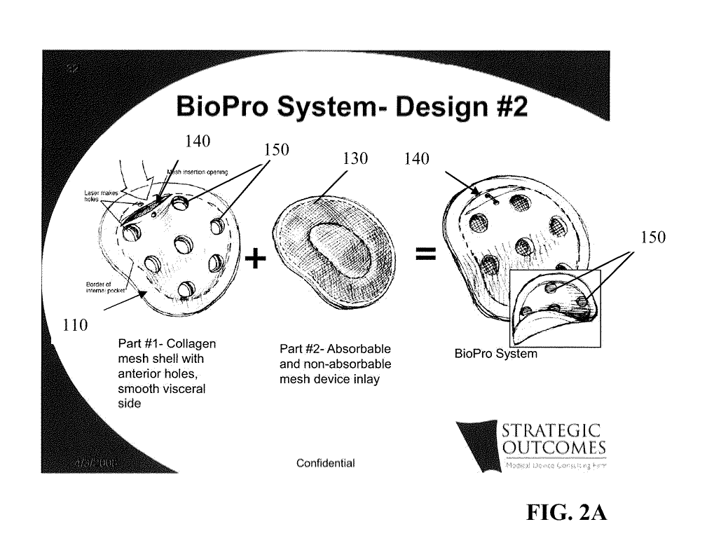

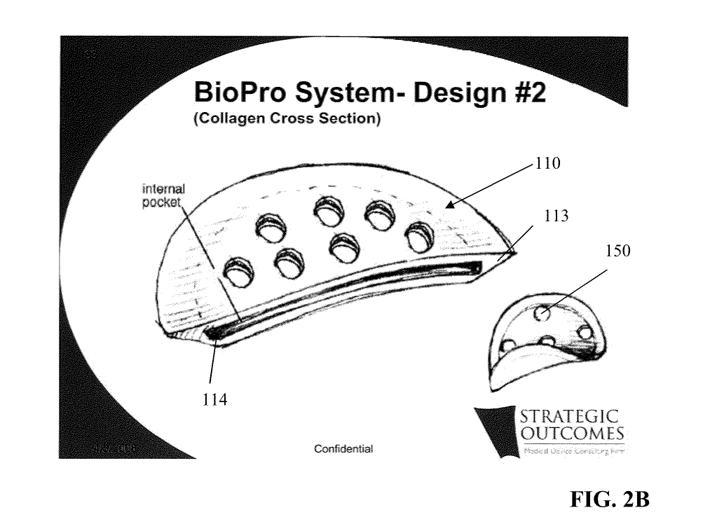

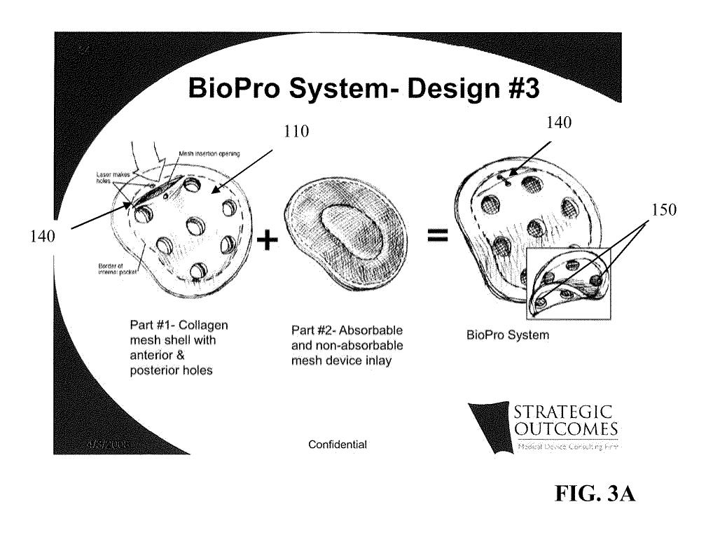

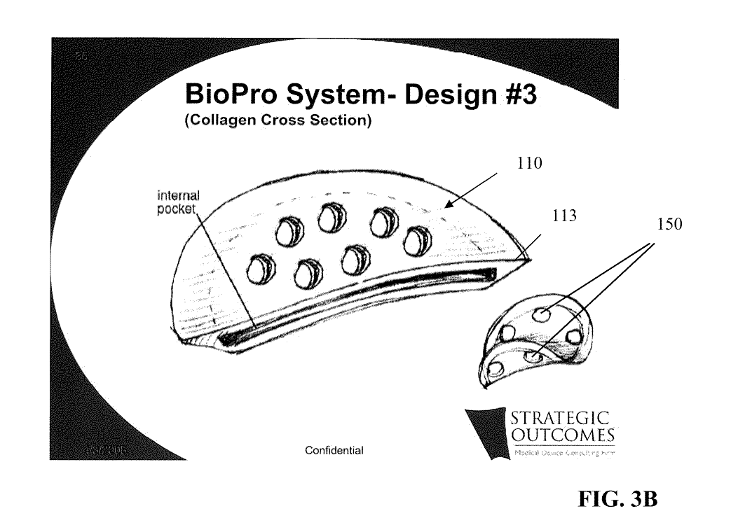

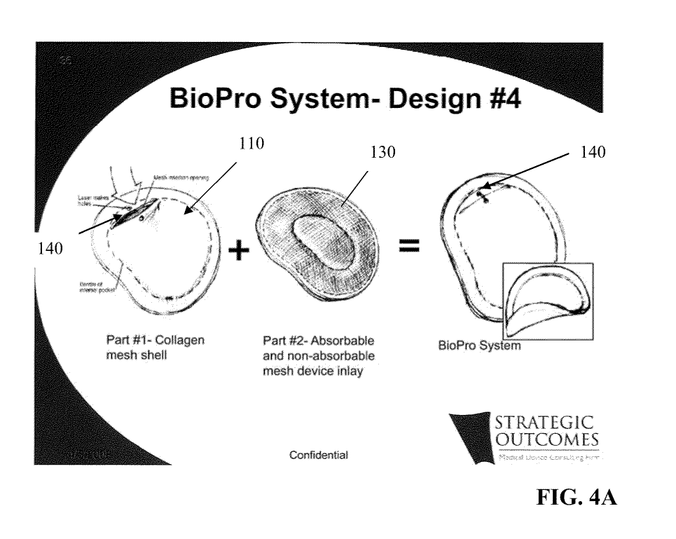

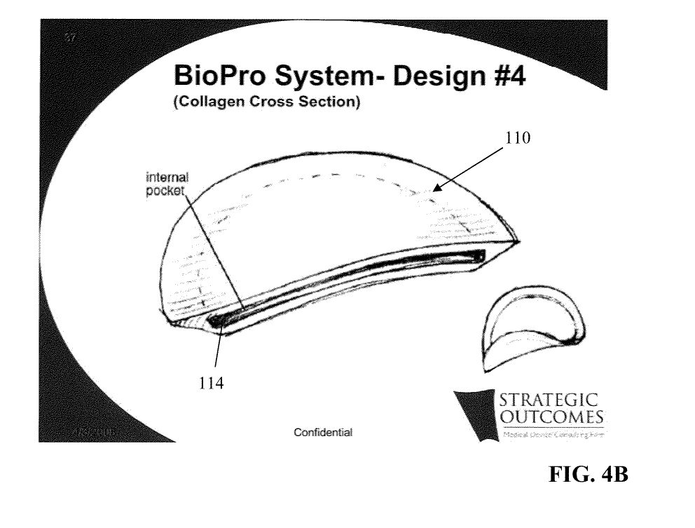

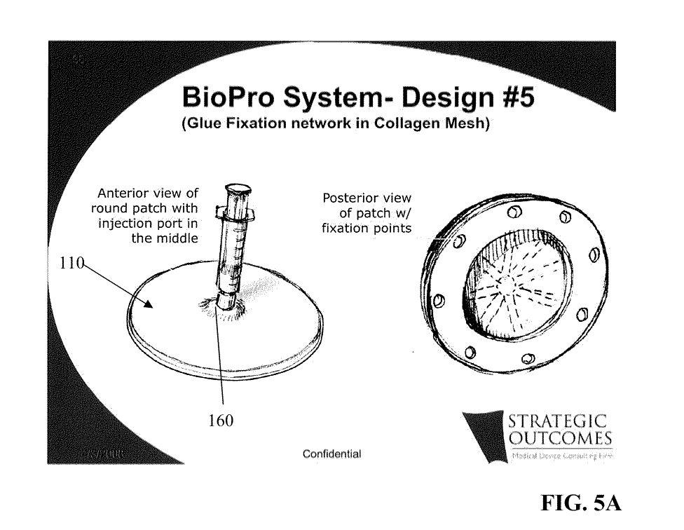

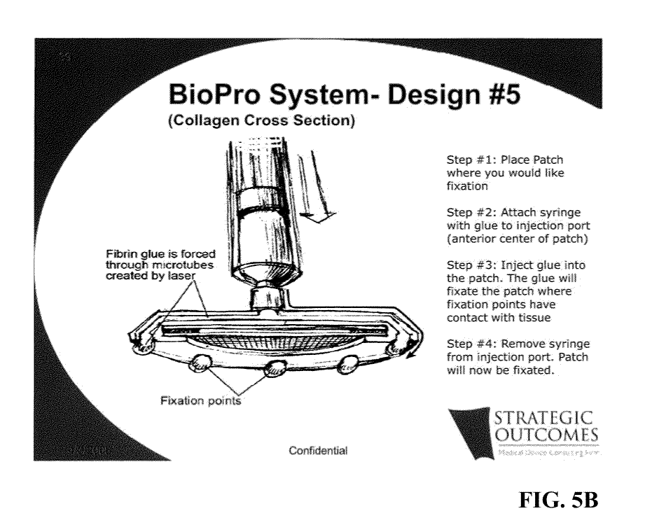

Opening 112 can be any suitable size and shape, including for example a wide opening covering at least 30%, 40%, or 50% of a side of the viscera attachment barrier 110 (see FIGS. 1A-1C), or a slit 140 (see FIGS. 2A-2B, 3A-3B, and 4A-4B), or even an injection port 160 (see FIGS. 5A-5B). The viscera attachment barrier 110 can advantageously include a plurality of fenestrations 150 (see FIGS. 2A-2B, and 3A-3B). The fenestrations 150 can be on the anterior side of the material (see FIGS. 2A-2B, posterior side of the material (not shown) or the anterior and posterior sides of the material (see FIGS. 3A-3B).

It is also contemplated that the shell 112 can have a tapered edge 113 (see FIGS. 1B, 1C, 2B, and 3B). When inserted in the peritoneal cavity, the angle of the taper is preferably selected to enhance growth of peritoneal cells over and around the edges of the shell. All suitable heights for the taper are contemplated, including especially heights in the range of 1-3 mm. Unless the context dictates to the contrary, all ranges set forth herein should be interpreted as inclusive of their endpoints.

Shell 112 can advantageously have fenestrations 150 that will allow the non-absorbable mesh to make direct contact with tissue to promote immediate, reliable in-growth.

It is also contemplated that shell 150 can have fixation openings that facilitate the fixation of the viscera separating barrier to the absorbable mesh a non-absorbable mesh or an absorbable and non-absorbable mesh combination. These openings can or can not be limited to the anterior portion of the patch as to not disturb the smooth profile of the viscera separating posterior portion of the patch. By utilizing these openings the prosthesis layers can be coupled by gluing, sewing or mechanical means; tacks, clips, crimping.

It is also contemplated that the inside of the viscera separating barrier pocket can contain “loop holes or tags” 170 that facilitate the fixation mesh to the shell medial to the peripheral edge. These “loop holes or tags” allow coupling without having to go all the way through the viscera separating barrier assuring that the smooth profile of the viscera separating posterior portion of the patch is not disturbed.

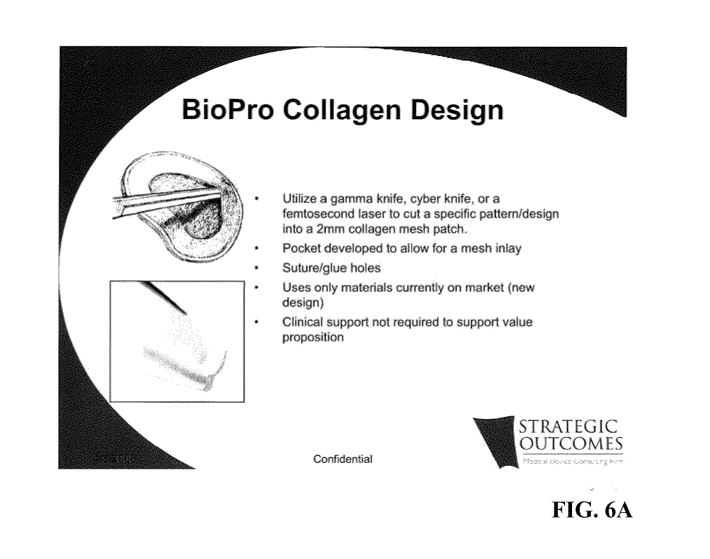

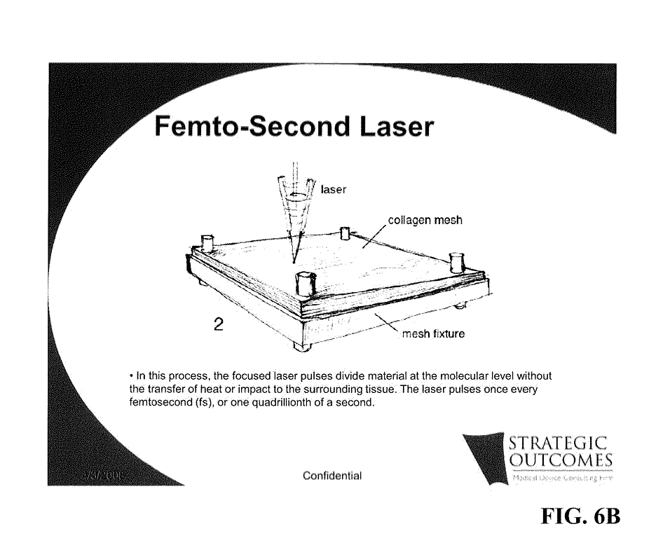

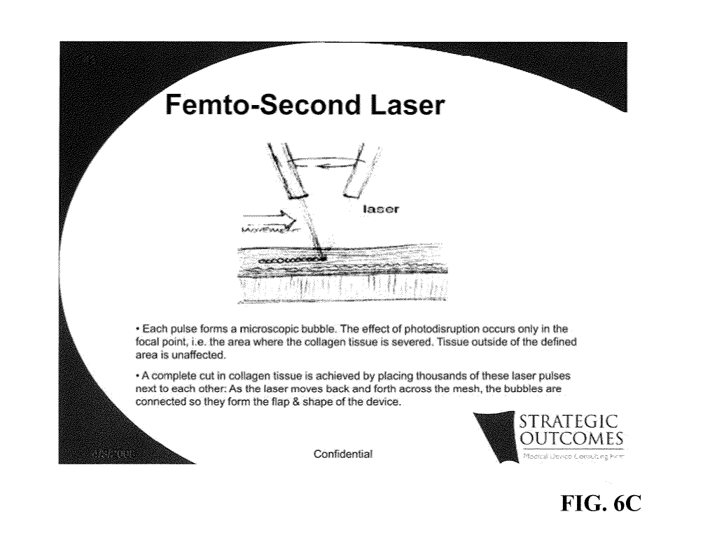

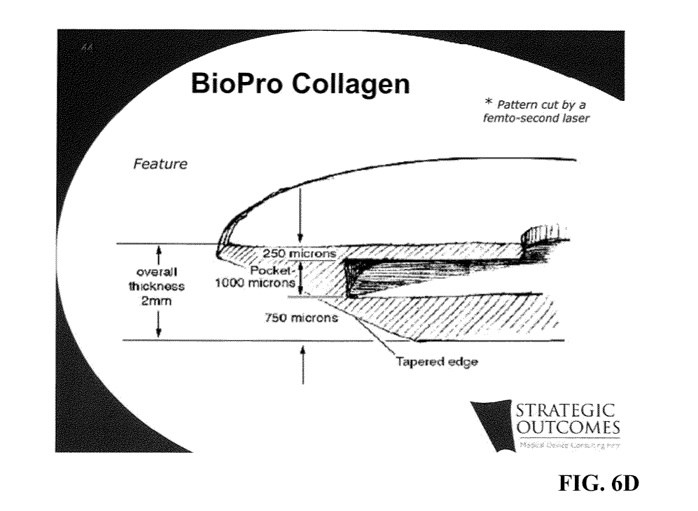

FIGS. 6A-6D show contemplated manufacturing details. The pocket in the shell can be developed by use of a femtosecond laser, gamma knife or any other suitable technology for making micro-incisions into materials. The techniques used to make the pocket can also be used to create a macroporous or microporous structure on the anterior or posterior side of the shell. Production of such macroporous or microporous structures is contemplated to be facilitated (quicker and stronger incorporation) through use of a highly cross linked collagen material for the shell. The same techniques used to make the pocket can be used to create a microtube network that can be used to help fixate the patch via glue (see FIGS. 5A-5B).

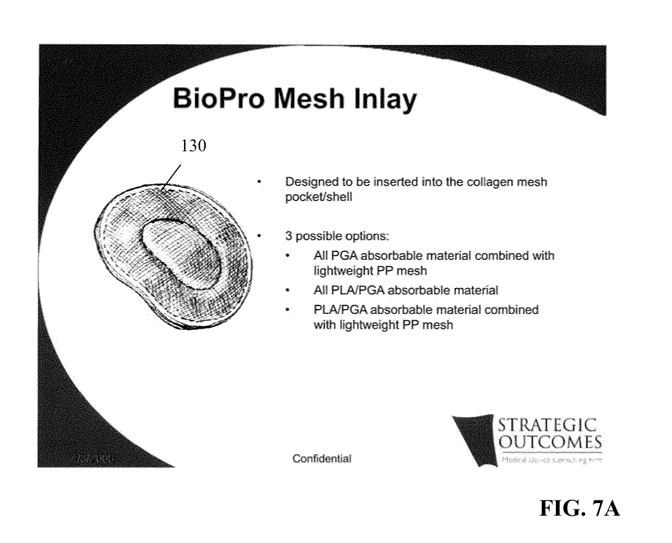

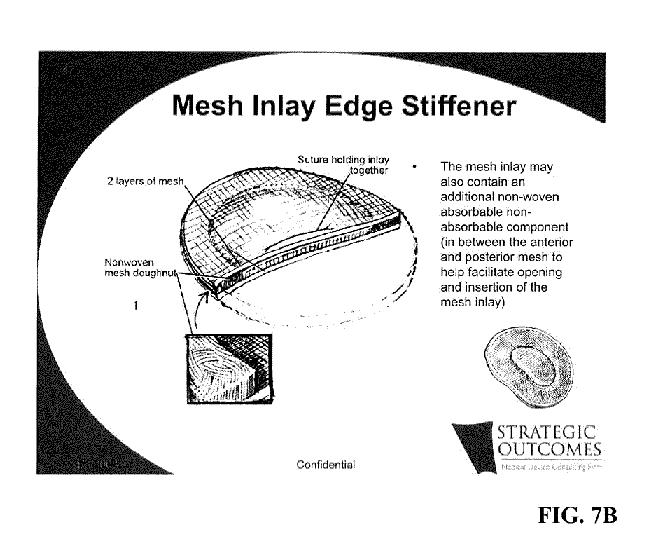

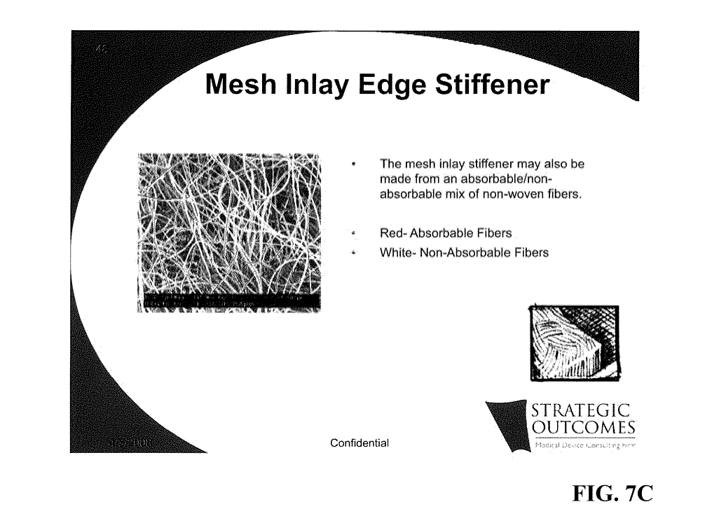

FIG. 7A show various preferred options of the insert 130 (i.e. mesh inlay) that is inserted into the shell. FIGS. 7B-7C show an optional edge stiffener for the insert. It is contemplated that the mesh inlay can comprise any suitable combination of layers, including for example two layers of absorbable mesh, two layers of non-absorbable mesh, or an absorbable and non-absorbable mesh combination. A “stiffener” can be placed between the two mesh layers (see FIG. 7B). This “stiffener” is preferably constructed of a non woven mesh network that can be non-absorbable, absorbable or an absorbable and non-absorbable combination. The non-woven material is contemplated to bring stability to the prosthesis and facilitate placement of the inlay into the viscera separating shell. The stiffener can cover some or all of the mesh inlay area. The inlay can be coupled together using any suitable mechanical or chemical techniques, including for example suture, glue, compression, etc. The stiffener can also be made from a silicone or other polymer material.

The mesh inlay can advantageously include macropores (greater than 0.2 mm wide) to allow for anterior and posterior portions of the shell to contact. In the scenario of using collagen mesh as the shell, the contact points will allow vessel growth and vascularization through and around the mesh.

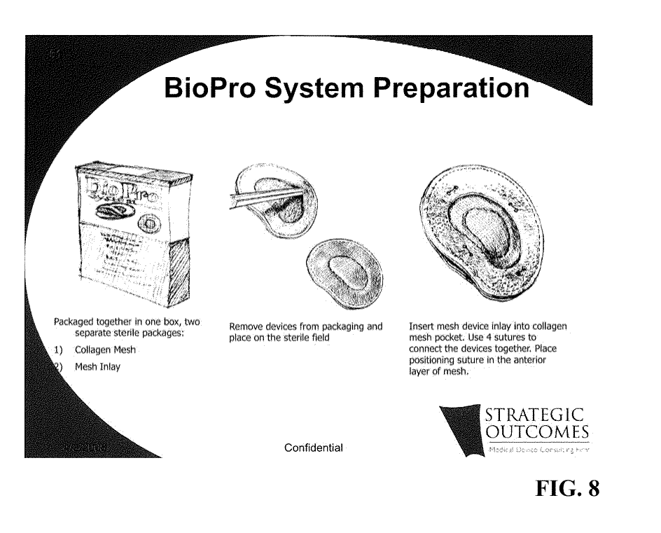

FIG. 8 shows a kit and corresponding instructions that can be used in a marketing program. The kit will preferably include both the inlay shell. Within a given kit these can be packaged separately or together. As used herein the term “instructions” should be interpreted broadly to include written and/or oral communications, which can be words, images, video, or any combination of these. The instructions need not be present on or in the kit.

It should be appreciated that one or more aspects of inserting at least one of (a) an absorbable mesh, (b) a non-absorbable mesh and (c) an absorbable and non-absorbable mesh combination into the shell can occur at any suitable stage, including for example during manufacturing prior to sale, and proximate (within a few hours of the beginning) of the relevant surgical procedure. It is especially contemplated that the insertion step can occur just prior (within 5, 10, 15, 30, 45, or 60 minutes) of implantation in the patient.

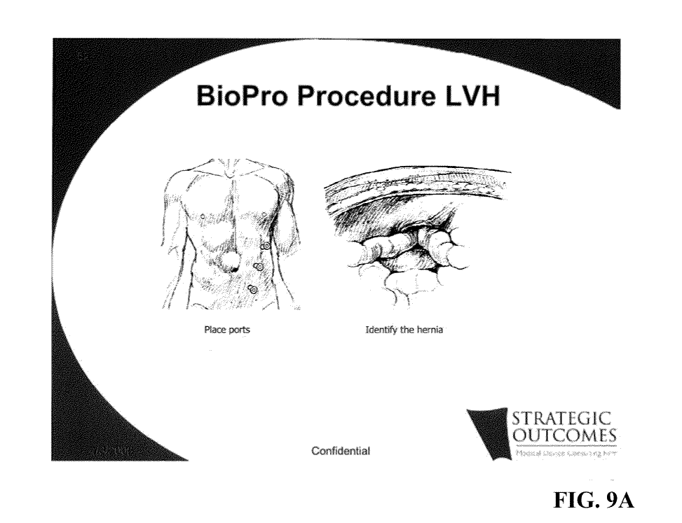

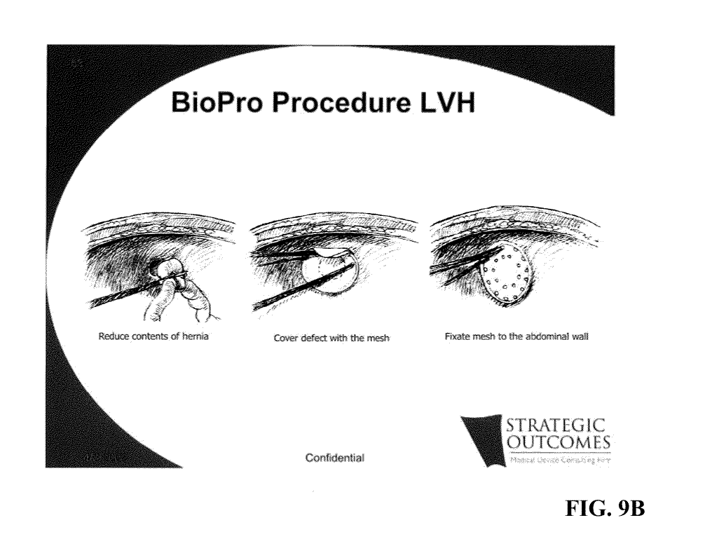

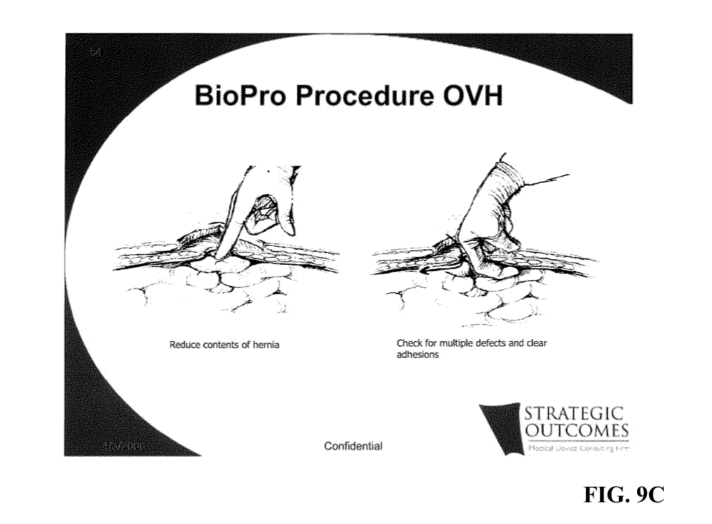

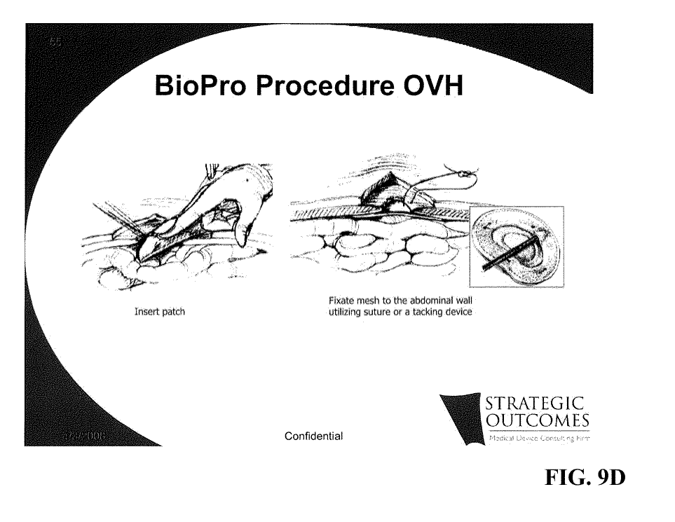

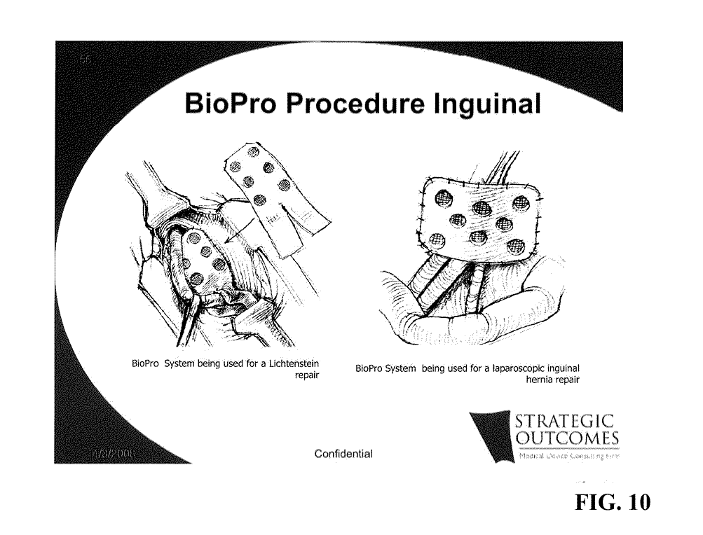

FIGS. 9A-9B depict steps in treatment of a Laparoscopic Ventral Hernia (LVH) and FIGS. 9C-9D depict steps in treatment of an Open Ventral Hernia Repair (OVH) using devices and methods disclosed herein. FIG. 10 depicts steps in inguinal hernia repair using devices and methods disclosed herein.

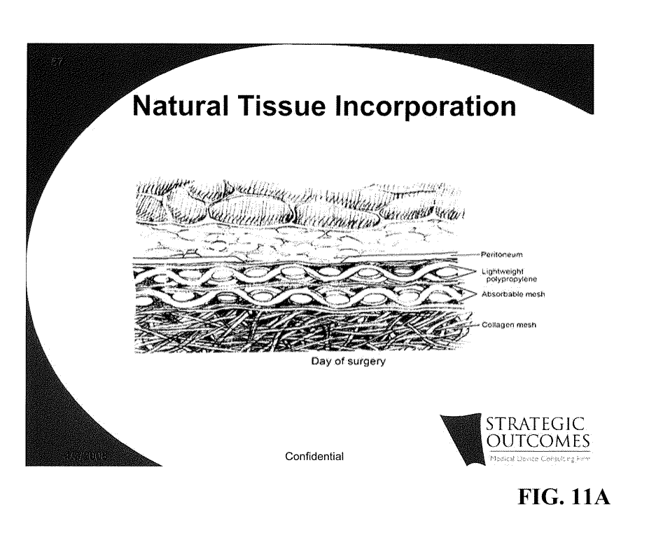

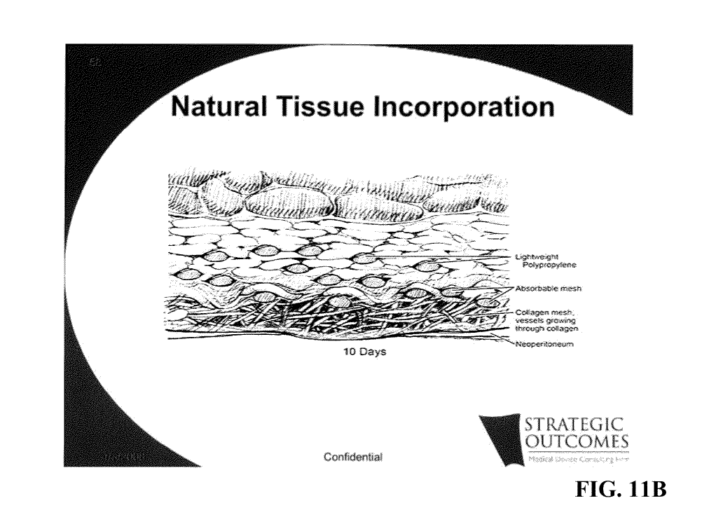

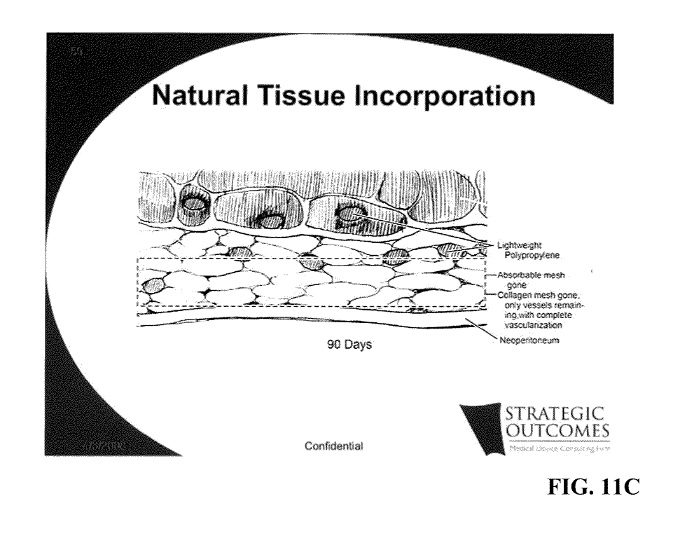

FIGS. 11A-11C depicts contemplated progression of tissue growth into a prosthesis as disclosed herein.

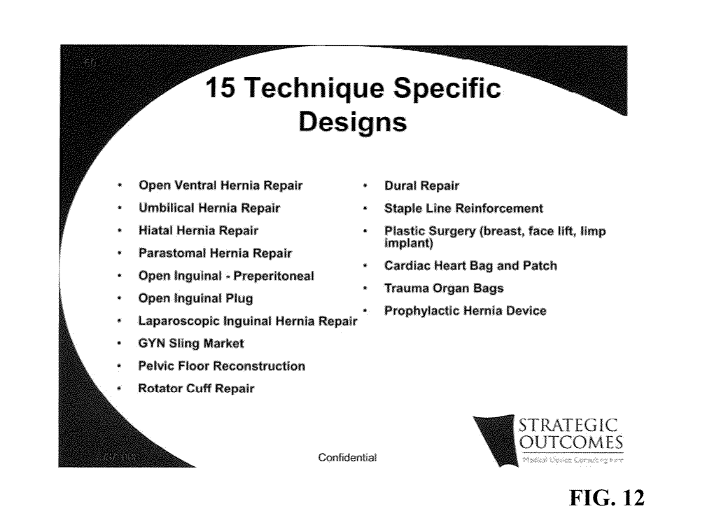

FIG. 12 is a listing of 15 procedures for which specific prosthesis designs are contemplated.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc. Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

What is claimed is:

1. An implantable prosthesis comprising:
a viscera separating barrier that defines a shell having a pocket that receives an insert; and
wherein in the insert comprises at least one of (a) a non-absorbable tissue infiltration material, (b) an absorbable tissue infiltration material, and (c) a combination non-absorbable and absorbable tissue infiltration material.

2. The prosthesis of claim 1 wherein the viscera attachment barrier comprises a collagen mesh.

3. The prosthesis of claim 1 wherein the viscera attachment barrier has a plurality of fenestrations.

4. The prosthesis of claim 2 wherein the non-absorbable tissue infiltration material comprises at least one of a polypropylene mesh, a polyester, or a PTFE.

5. The prosthesis of claim 2 wherein the absorbable mesh comprises at least one of a polyglactin, a polyglycolic acid (PGA), a polyglactin, a polylactic acid (PLA).

6. The prosthesis of claim 1 wherein the non-absorbable tissue infiltration material and the absorbable mesh are coupled together to form the insert.

7. The prosthesis of claim 1 wherein the insert further includes a stiffener.

8. The prosthesis of claim 1 wherein the pocket has an opening comprising at least 30% of an area of a side of the shell.

9. The prosthesis of claim 1 wherein the shell has a slit opening sized and dimensioned to receive the insert.

10. The prosthesis of claim 1 wherein the shell has a tapered edge having a height of 1-3 mm.

11. A method of treating a patient, comprising:
providing a viscera attachment barrier that defines a shell having a pocket with an opening;
inserting at least one of (a) an absorbable mesh, (b) a non-absorbable mesh, and (c) an absorbable and non-absorbable mesh combination into the opening; and
implanting the shell into the patient.

12. The method of claim 11 further comprising providing the shell with a plurality of fenestrations.

13. The method of claim 11, further comprising providing the absorbable mesh as an insert coupled to a non-absorbable mesh.

14. The method of claim 11, wherein the step of inserting is at least partially accomplished proximate a surgical procedure or prior to implantation in the patient.

15. The method of claim 11, further comprising selling a package containing the shell into which has been inserted at least one of (a) the absorbable mesh, (b) the non-absorbable mesh and (c) the absorbable and non-absorbable mesh combination.

16. A method of manufacturing an implantable patch, comprising:
providing a viscera attachment barrier that defines a shell having a pocket with an opening; and
providing in combination with the barrier an insert that includes at least one of (a) an absorbable mesh, (b) a non-absorbable mesh and (c) an absorbable and non-absorbable mesh combination, the insert sized and dimensioned to be inserted into opening.

17. The method of manufacturing of claim 16, further comprising introducing a plurality of macro holes into the barrier.

18. The method of manufacturing of claim 16, further comprising providing the insert with a plurality of micro tubes.

19. The method of manufacturing of claim 18, wherein the step of providing the insert with a plurality of micro tubes comprises including the micro tubules in a glue used to manufacture the insert.

20. The method of manufacturing of claim 18, wherein the step of providing the insert with a plurality of micro tubes comprises including the micro tubules into the mesh of the insert.
